(19) United States
(12) Patent Application Publication
(10) Pub. No.: US 2026/0337140 A1
(43) Pub. Date: Oct. 29, 2026

(54) METHOD FOR PRODUCING BIOBASED a,B-UNSATURATED CARBOXYLIC ACIDS FROM POLY(3-HYDROXYALKANOATE) CONTAINED IN BIOMASS

(71) Applicant: Arkema France, Puteaux (FR)

(72) Inventors: Serge TRETJAK, Saint Avold Cedex (FR); Yves CABON, Saint Avold Cedex (FR); Julien JOUANNEAU, Pierre-Benite Cedex (FR)

(21) Appl. No.: 19/471,861

(22) PCT Filed: Mar. 29, 2024
(86) PCT No.: PCT/FR2024/050412

§ 371 Date: Oct. 2, 2025

(30) Foreign Application Priority Data
Apr. 5, 2023 (FR) FR23.03387

(51) Int. Cl. C07C 51/09; C07C 51/44

(52) U.S. Cl. CPC C07C 51/09; C07C 51/44

(57) ABSTRACT

The present invention relates to a process for producing biobased α,β-unsaturated carboxylic acids from a biomass containing a poly(3-hydroxyalkanoate), comprising a step of thermolysis of said biomass, in the presence of polymerization inhibitors and in the absence of catalyst, followed by several steps leading to the production of α,β-unsaturated carboxylic acids.

### TECHNICAL FIELD

The present invention relates to a process for producing biobased α,β-unsaturated carboxylic acids from a biomass containing a poly(3-hydroxyalkanoate) in the presence of polymerization inhibitors, comprising a step of thermolysis of said biomass, in the absence of catalyst, followed by several steps leading to the production of α,β-unsaturated carboxylic acids.

### PRIOR ART AND TECHNICAL PROBLEM

α,β-Unsaturated carboxylic acids are nowadays produced industrially mainly from feedstocks of fossil origin. For example, acrylic acid is obtained by oxidation of propylene, or methacrylic acid can be obtained by oxidation of isobutylene.

One possible way of obtaining these α,βunsaturated carboxylic acids is the thermolysis at temperatures of 150 °C to 300 °C of the corresponding poly(3-hydroxyalkanoates) (P3HA), according to the following reaction:

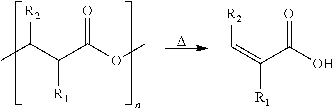

R₁═H or alkyl and R₂═H or alkyl; n is a number greater than 30
If R₁═R₂═H:
   Poly(3-hydroxyalkanoate)=poly(3-hydroxypropionate) (P3HP);
   α,β-Unsaturated carboxylic acid=propenoic acid (acrylic acid).
If R₁=methyl and R₂═H:
   Poly(3-hydroxyalkanoate)=poly(3-hydroxyisobutyrate) (P3HiB);
   α,β-Unsaturated carboxylic acid=isobutenoic acid (methacrylic acid).
If R₁═H and R₂=methyl:
   Poly(3-hydroxyalkanoate)=poly(3-hydroxybutyrate) (P3HB);
   α,β-Unsaturated carboxylic acid=but-2-enoic acid (crotonic acid).
If R₁═H and R₂=ethyl:
   Poly(3-hydroxyalkanoate) is poly(3-hydroxyvalerate) (P3HV);
   α,β-Unsaturated carboxylic acid=pent-2-enoic acid

These poly(3-hydroxyalkanoates) can themselves be obtained beforehand by chemical transformations of feedstocks of fossil origin, but also by fermentation of biomass.

There is strong market demand for these α,β-unsaturated carboxylic acids, which are used as monomers in numerous applications, to be obtained from biobased feedstocks. These biobased feedstocks are derived from renewable organic matter (biomass) of biological origin (microorganisms, plants or animals).

A potential problem with such a process is that the P3HA obtained by fermentation is present inside the cell. Thermolysis is therefore carried out in the presence of the cell membrane, which poses problems of fouling of the reactor or the presence of impurities in the end product.

A number of solutions to this problem have been proposed.

Document U.S. Pat. No. 9,850,192 describes a process for producing acrylic acid from a genetically modified microbial biomass metabolizing glucose or any other renewable feedstock to produce a poly-3-hydroxypropionate (P3HP) homopolymer or copolymer inside microbial cells. Said process comprises a step of thermolysis of the washed/dried/milled P3HP-containing biomass in the presence of a catalyst. This process actually makes it possible to produce acrylic acid while limiting the formation of acrylic acid oligomers, such as the acrylic acid dimer which is spontaneously formed during the production of acrylic acid. Acrylic acid is recovered in gaseous form and then condensed, while the catalyst and the residual mass of biomass can be recycled into the process or subjected to thermal regeneration. However, there is a risk that the residue present in the reactor after thermolysis will be pasty and tacky, which could complicate its transfer to an industrial scale. Example 5 and FIG. 7 describe how to carry out this invention on an industrial scale. After fermentation, the biomass is washed, and dried using either an atomizer or a double-drum dryer. After addition of the catalyst, the product is pyrolyzed in a FAST™ reactor at 250-350 °C with a residence time of between 0.25-1 hour using an inert gas such as nitrogen to send the vapors formed to the purification equipment. The vapor phase is composed of 90% organic/water and 10% inert gas. The gas is then purified, according to the process described in document U.S. Pat. No. 6,646,161 or in document US 20120006673, to obtain acrylic acid still containing many impurities. Complete purification is carried out using distillation columns, as described in documents U.S. Pat. Nos. 7,332,624 and 7,179,875, and may also require crystallization operations, as described in documents U.S. Pat. Nos. 6,482,981 and 71,798,750.

Another solution consists in first extracting the P3HA from the biomass using an organic solvent before subjecting it to thermolysis. Document US 20150376152 describes in example 6 the extraction of P3HP from biomass, using an organic solvent, such as 2-butanone, then the production of acrylic acid in three steps: evaporation of the solvent and condensation of the latter in a receiving vessel; thermal degradation of the P3HP in the absence of inhibitor leading to the production of acrylic acid vapor, and finally distillation and condensation of the acrylic acid in a receiving vessel containing hydroquinone to prevent the polymerization of the acrylic acid.

In their application FR 2208914, the applicant company proposes to carry out the thermolysis of the P3HA in the absence of catalyst and in the presence of a polymerization inhibitor; characteristically, the vapor pressure is less than twice the vapor pressure of one of the inhibitors at the thermolysis temperature, which has the effect of preventing the formation of polymers in the reactor and also in the gas phase in the event of accidental condensation or at the time the acrylic acid vapors condense at the top of the column.

In their application FR 2208916, the applicant company describes a process using a solvent which makes it possible, starting from biomass, to selectively solubilize the P3HA, to separate the organic detritus insoluble in said solvent, in order to carry out a thermolysis treatment in the liquid phase in the presence of polymerization inhibitors.

Document WO 2016/039618 describes the thermal degradation of a dry biomass containing poly(3-hydroxybutyrate) in order to produce crotonic acid. In example 1, it is shown that crotonic acid can be obtained with comparable yields of less than 60% from wet or dry biomass, in the absence of catalyst.

It has now been discovered that it is possible to simplify the procedure for the production of α,β-unsaturated carboxylic acids, by carrying out the P3HA thermolysis operation directly on biomass mixed with at least one polymerisation inhibitor, without prior separation of the cell membrane, without needing a solvent to carry out the thermolysis, without using a catalyst, and without injecting inert gas to entrain the vapors out of the reaction zone.

More precisely, “in the absence of catalyst” is understood to mean that the thermolysis of the PHA in the presence of at least one polymerization inhibitor takes place in the absence of another chemical species that accelerates or reorients the reaction kinetics. The thermolysis as envisaged is induced only by operating conditions such as temperature, pressure and residence time.

Actually excluded are the chemical species that lead to different types of catalysis depending on the nature of the species:
homogeneous catalysis, if the catalyst and the reactants form only one phase (often a liquid phase);
heterogeneous catalysis, if the catalyst and the reactants form several phases (usually a solid catalyst for gas-phase or liquid-phase reactants);
enzymatic catalysis, if the catalyst is an enzyme, i.e. a protein.

The invention accordingly proposes to provide a simple and easily implementable solution for reducing fouling phenomena and the presence of impurities in the end product and thus maintain high reliability and increased productivity in processes for producing α,β-unsaturated carboxylic acids from poly(3-hydroxyalkanoates) obtained by fermentation.

### SUMMARY OF THE INVENTION

The present invention relates to a process for producing a biobased α,β-unsaturated carboxylic acid from a biomass containing a poly(3-hydroxyalkanoate) (P3HA) in the absence of catalyst, said process comprising the following steps:
mixing said biomass with at least one polymerization inhibitor;
subjecting said biomass-inhibitor solid mixture to a thermolysis step resulting in, firstly, said α,β-unsaturated carboxylic acid in the vapor phase and, secondly, a solid residue;
separating the two phases formed into a gas phase and a solid phase;
purifying said gas phase to obtain a purified α,β-unsaturated carboxylic acid;
treating the solid-phase residue.

According to various embodiments, said process comprises the following features, where appropriate in combination. The contents indicated are expressed by weight, unless otherwise mentioned. In the ranges of values indicated, the limits are included.

According to one embodiment, the poly(3-hydroxyalkanoate) contained in the biomass comprises a single type of 3-hydroxyalkanoate unit and the product formed is therefore composed of a single α,β-unsaturated carboxylic acid.

According to one embodiment, the poly(3-hydroxyalkanoate) is poly(3-hydroxypropionate) and the α,β-unsaturated carboxylic acid produced is acrylic acid.

According to one embodiment, the poly(3-hydroxyalkanoate) is poly(3-hydroxybutyrate) and the α,β-unsaturated carboxylic acid produced is crotonic acid.

According to one embodiment, the poly(3-hydroxyalkanoate) is poly(3-hydroxyisobutyrate) and the α,β-unsaturated carboxylic acid produced is methacrylic acid.

According to one embodiment, the poly(3-hydroxyalkanoate) contained in the biomass comprises a plurality of different 3-hydroxyalkanoate units and the product formed is therefore composed of a mixture of different α,β-unsaturated carboxylic acids. Examples of P3HA copolymers are poly-3-hydroxybutyrate-co-3-hydroxypropionate, poly-3-hydroxybutyrate-co-3-hydroxyvalerate (poly-3-HB-co-3HV).

According to one embodiment, the poly(3-hydroxyalkanoate) contains the 3-hydroxypropionate unit and at least one of the α,β-unsaturated carboxylic acids produced is acrylic acid.

According to one embodiment, the poly(3-hydroxyalkanoate) contains the 3-hydroxybutyrate unit and at least one of the α,β-unsaturated carboxylic acids produced is crotonic acid.

According to one embodiment, the poly(3-hydroxyalkanoate) contains the 3-hydroxyisobutyrate unit and at least one of the α,β-unsaturated carboxylic acids produced is methacrylic acid.

According to one embodiment, the biomass host is a bacterium, yeast, fungus, alga, cyanobacterium or a mixture of two or more of these elements.

The biomass is subjected to a thermolysis reaction, which takes place in the presence of one or more polymerization inhibitors.

According to one reaction mode, the biomass is intimately mixed with one or more polymerization inhibitors before thermolysis.

According to one embodiment, the process according to the invention comprises a step of condensing the vapors of the α,β-unsaturated carboxylic acid(s) obtained by the biomass thermolysis reaction, followed by one or more purification steps. The purification operations can generally include distillations, liquid/liquid extractions, separations using a film evaporator, or crystallizations.

According to one embodiment, the process according to the invention comprises a step of treating said residue obtained at the end of thermolysis, for example by upgrading the latter via hydrothermal gasification to give methane.

The present invention meets the need expressed in the prior art. It makes it possible to prevent the risks of fouling of the thermolysis reactor and of the gas phase in the event of accidental condensation, while making it possible to obtain a gas phase rich in one or more α,β-unsaturated carboxylic acids and also containing polymerization inhibitors and also a solid residue that can be upgraded.

This solution has two advantages: it saves having to distill off the solvent, which is energy-intensive, and the thermolysis is performed on the P3HA contained in the biomass, in the solid state and not in solution, which reduces the energy and environmental cost of the process.

The invention will now be described in more detail in the description that follows.

### DETAILED DESCRIPTION OF THE INVENTION

The aim of the invention is to produce biobased α,β-unsaturated carboxylic acids on an industrial scale by thermolysis of poly(3-hydroxyalkanoate) contained in the biomass, while limiting the problems of fouling of the thermolysis reactor and/or the presence of impurities in the end product.

The term “thermolysis” of poly(3-hydroxyalkanoate) (P3HA) means its chemical decomposition into α,β-unsaturated carboxylic acid that occurs under the effect of temperature. This term is synonymous with pyrolysis.

The present invention relates to a process for producing a biobased α,β-unsaturated carboxylic acids from a biomass containing a poly(3-hydroxyalkanoate) (P3HA) in the absence of catalyst, said process comprising the following steps:
mixing said biomass with at least one polymerization inhibitor, in the solid state;
subjecting said biomass-inhibitor solid mixture to a thermolysis step resulting in, firstly, said α,β-unsaturated carboxylic acid in the vapor phase and, secondly, a solid residue;
separating the two phases formed into a gas phase and a solid phase;
purifying said gas phase to obtain a purified α,β-unsaturated carboxylic acid;
treating the solid-phase residue.

According to one embodiment, said process for producing biobased α,β-unsaturated carboxylic acids from a biomass containing a poly(3-hydroxyalkanoate) (P3HA) comprises the following steps:
introducing the biomass (in powder form) and at least one polymerization inhibitor into a (solid-phase) mixer by means of a pipe or by an endless screw-type conveyor;
mixing the biomass and at least one polymerization inhibitor in a conveyor mixer comprising several endless screws actuated in a sheath or directly in a reactor known as a thermolysis reactor;
thermolyzing this mixture at a given temperature and at a controlled pressure in a system suitable for treating the molten or pasty residue, so as to generate a vapor phase and a pasty and/or solid phase;
separating the two phases formed in a gas-liquid separator;
treating the residue for upgrading by land spreading, combustion or hydrothermal gasification;
condensing the gas phase, carried out by successive condensation temperature adjustment by placing one or more condensers in series and separating the resulting gas and liquid phases containing α,β-unsaturated carboxylic acid and contaminants which can be recycled back into the reactor or sent to the purification system;
treating the condensed phase to obtain the α,β-unsaturated carboxylic acid by using one to several distillation columns making it possible, firstly, to separate the α,β-unsaturated carboxylic acid from the products heavier than the latter and, secondly, to obtain products lighter than the latter;
purifying the resulting α,β-unsaturated carboxylic acid by a liquid/solid separation method such as crystallization, or by a gas/liquid separation method such as distillation.

The invention is based on the use of a mixture of biomass containing a P3HA and at least one polymerization inhibitor by performing a technique for mixing solids and heat treating this mixture.

The term “biomass” means organic matter originating from plants (including microalgae), animals, bacteria or fungi that is employable as a source of biobased feedstocks, as opposed to feedstocks of fossil origin.

In the process according to the invention, the first step uses genetically modified host biomass derived from genetic engineering. According to one embodiment, the biomass host is a bacterium, yeast, fungus, alga, cyanobacterium or a mixture of two or more of these elements.

The biomass is obtained by a prior step of culturing a recombinant host with a renewable feedstock. According to one embodiment, the renewable feedstock is selected from glucose, fructose, sucrose, arabinose, maltose, lactose, xylose, ethanol, methanol, glycerol, fatty acids, vegetable oils and biomass-derived synthesis gas or a combination thereof.

According to one embodiment, the biomass used in the process according to the invention comes from a process of bacterial fermentation of sugars or lipids.

Depending on the culture conditions and on the variety of microorganism used, poly(3-hydroxyalkanoate) (P3HA) homo- or copolymers with different 3-hydroxyalkanoic acids are formed.

The process according to the invention advantageously comprises a prior biomass preparation step, in which the biomass is treated by washing, drying and milling operations, to give a solid biomass (for example in powder form) containing at least 30% by weight of P3HA, preferably at least 50% by weight of P3HA.

The biomass is then mixed with at least one polymerization inhibitor chosen from the inhibitors conventionally used in existing industrial processes for the production of α,β-unsaturated carboxylic acids. These include phenol derivatives such as hydroquinone (HQ) and derivatives thereof such as hydroquinone methyl ether (MEHQ), 2,6-di-tert-butyl-4-methylphenol (BHT) or 2,4-dimethyl-6-tert-butylphenol (Topanol A); phenothiazine and derivatives thereof; nitroxide compounds such as 4-hydroxy-2,2,6,6-tetramethylpiperidin-1-oxyl (4-OH-TEMPO); and amino compounds such as para-phenylenediamine derivatives.

According to a preferred embodiment, at least one of said polymerization inhibitors is hydroquinone methyl ether (MEHQ).

According to one embodiment, the weight content of inhibitor in the mixture is between 0.1% and 10%, preferably from 1% to 5%.

According to one embodiment, the mixing and thermolysis steps are carried out continuously.

The mixture of the biomass and at least one polymerisation inhibitor is then subjected to thermolysis in the solid or partially molten state.

Thermolysis of the biomass-inhibitor mixture is carried out under temperature and pressure conditions allowing the chemical decomposition of the P3HA and the generation of one or more α,β-unsaturated carboxylic acids in the gaseous state.

According to one embodiment, the system for feeding the biomass and at least one polymerization inhibitor may be a pipe, an endless screw, a conveyor belt or a hopper, a pneumatic conveyor, a vibrating conveyor or an extruder. In addition, they may be coupled to a metering device.

The system according to the invention for heating the biomass-inhibitor mixture also comprises a reactor suitable for the heating for the purpose of the thermolysis of P3HA. For example, the heating can be carried out by exposing the mixture to microwaves, to pulsed electric fields or to a preheated inert gas or steam, with a preheated solid such as sand, by contact with a hot surface for instance in an extruder, a screw conveyor, a rotating drum or a platen. The hot surface can be heated by various means: direct electric heating, heating by heat transfer fluid (steam, oil, molten salts).

According to one embodiment, the heat is supplied through a hot surface heated by a heat-transfer fluid and in particular pressurized steam.

The reactor can be a conveyor but also any type of dryer known to those skilled in the art, described for example on the site:

https://www.techniques-ingenieur.fr/base-documentaire/42665210-production-des-medicaments-industrialisation/download/i2455/sechage-industriel.html

According to one embodiment, said reactor is a reactor suitable for pyrolysis, for high-temperature pyrolysis, or a fluidized reactor or a reactor suitable for solvolysis or else a reactor consisting of hollow plates heated by a heat-transfer fluid circulating in the plates. However, reactors enabling higher gains in yield of acrylic acid have been identified, such as: a conveyor, a rotating drum and/or a set of heating plates.

According to one embodiment, said reactor is a device of mixer-conveyor type, for example of paddle dryer type. This device comprises a reactor in which rotating blades or paddles are arranged. The paddle makes it possible to mix and homogenize the incoming feeds of biomass and polymerization inhibitor. This mixer-conveyor also has the advantage of enabling large amounts of mixture to be treated. It also enables good heat transfer between the wall and the mixture. Such a device is customarily used at a moderate temperature for drying a solid, but, in the context of the invention, by increasing the temperature, it is possible to carry out the thermolysis.

According to one embodiment, said reactor is a device of mixer-conveyor type, for example of screw conveyor type. This device comprises a reactor in which two endless screws operate in opposition. The mixture is heated through the hot wall using a heat-transfer fluid such as steam. The movement of the two screws makes it possible to mix and homogenize the incoming feeds of biomass and polymerization inhibitor and then, by increasing the temperature, to carry out the thermolysis.

According to one embodiment, the temperature in the thermolysis reactor is between 100 °C and 250 °C, preferably between 150 °C and 200 °C, in order to enable the thermolysis of the P3HA. This temperature can also be monitored by means of temperature sensors placed in the mixer. Such moderate heating allows all or some of the mixture to liquefy while at the same time preventing polymerization of the α,β-unsaturated carboxylic acid.

According to one embodiment, the pressure in the thermolysis reactor is between 3 kPa and 101 kPa, preferably between 15 kPa and 40 kPa.

According to one embodiment, the residence time in the thermolysis reactor is between 0.5 h and 5 h, preferably between 2-4 h.

The mixing between the biomass and the inhibitor, and the thermolysis reaction can be carried out consecutively or simultaneously.

In the thermolysis reactor, the biomass-inhibitor mixture is converted under the action of heat into gaseous compounds comprising one or more α,β-unsaturated carboxylic acids.

According to one embodiment, the poly(3-hydroxyalkanoate) contained in the biomass comprises a single type of 3-hydroxyalkanoate units and the product formed is therefore composed of a single α,β-unsaturated carboxylic acid.

According to one embodiment, the poly(3-hydroxyalkanoate) contained in the biomass is poly(3-hydroxypropionate) and the α,β-unsaturated carboxylic acid produced is acrylic acid.

According to one embodiment, the poly(3-hydroxyalkanoate) contained in the biomass is poly(3-hydroxybutyrate) and the α,β-unsaturated carboxylic acid produced is crotonic acid.

According to one embodiment, the poly(3-hydroxyalkanoate) contained in the biomass is poly(3-hydroxyisobutyrate) and the α,β-unsaturated carboxylic acid produced is methacrylic acid.

According to one embodiment, the poly(3-hydroxyalkanoate) contained in the biomass comprises a plurality of different 3-hydroxyalkanoate units and the product formed is therefore composed of a mixture of different α,β-unsaturated carboxylic acids. Examples of P3HA copolymers are poly-3-hydroxybutyrate-co-3-hydroxypropionate, poly-3-hydroxybutyrate-co-3-hydroxyvalerate (poly-3-HB-co-3HV).

According to one embodiment, the poly(3-hydroxyalkanoate) contained in the biomass contains the 3-hydroxypropionate unit and at least one of the α,β-unsaturated carboxylic acids produced is acrylic acid.

According to one embodiment, the poly(3-hydroxyalkanoate) contained in the biomass contains the 3-hydroxybutyrate unit and at least one of the α,β-unsaturated carboxylic acids produced is crotonic acid.

According to one embodiment, the poly(3-hydroxyalkanoate) contained in the biomass contains the 3-hydroxyisobutyrate unit and at least one of the α,β-unsaturated carboxylic acids produced is methacrylic acid.

According to one embodiment, the process according to the invention makes it possible to manufacture several biobased α,β-unsaturated carboxylic acids from the poly(3-hydroxyalkanoate)s contained in the biomass.

Gases containing the α,β-unsaturated carboxylic acid(s) can be sent to a cooling system in order to be condensed. The condensate obtained may then be collected in a chamber intended for this purpose. The reactor enclosure and also the chamber are preferably under reduced pressure. The condensation system may be equipped with an injection of one or more inhibitors. In order to enable the recovery of one or more purified α,β-unsaturated carboxylic acids, the system may comprise a purification device, for example one or more distillation columns, or one or more items of liquid extraction, crystallization or membrane separation equipment.

The solid residue is then upgraded, for example by hydrothermal gasification or in the form of energy by combustion.

According to one embodiment, the condensation is carried out by successive pressure adjustment and separation of the resulting gas and liquid phases containing α,β-unsaturated carboxylic acid and contaminants which can be recycled back into the reactor.

According to the invention, the condensation is carried out by successive condensation temperature adjustment carried out by placing one or more tubular or spiral condensers in series and separation of the resulting gas and liquid phases containing α,β-unsaturated carboxylic acid and contaminants which can be recycled back into the reactor or sent to the purification system.

According to one embodiment, one or more polymerization inhibitors are added to the condenser.

According to one embodiment, there is no addition of inhibitor to the condenser.

According to one embodiment, this condensation can be carried out by bringing the α,β-unsaturated carboxylic acid in the gaseous state into contact with one or more α,β-unsaturated carboxylic acids in the liquid state. This contacting operation may be performed, for example, in a device of shower type, by spraying one or more liquid α,β-unsaturated carboxylic acids into a chamber collecting these α,β-unsaturated carboxylic acids in the gaseous state.

According to one embodiment, the condensation of the gas phase is carried out by a system of at least one condenser of the tubular condenser type at the pressure of the thermolysis, by cooling it and collecting the liquid phase obtained in a stirred storage tank, optionally with the addition of one or more additional inhibitors.

According to one embodiment, said products heavier than the α,β-unsaturated carboxylic acid are recycled upstream of the thermolysis reactor, or are mixed with the solid residue resulting from the thermolysis.

According to one embodiment, said products lighter than the α,β-unsaturated carboxylic acid are upgraded by combustion or gasification.

According to one embodiment, the residue obtained after the thermolysis step is upgraded by hydrothermal gasification.

According to the embodiment, the hydrothermal gasification is carried out at a temperature of 350 °C-450 °C and a pressure of 25 MPa.

According to one embodiment, the α,β-unsaturated carboxylic acid obtained is purified by a fractional crystallization operation comprising several separation stages to obtain high purity α,β-unsaturated carboxylic acid and a residue to be upgraded as energy.

The examples below illustrate the present invention without, however, limiting the scope thereof.

### EXPERIMENTAL SECTION

Example 1: The aim of this laboratory-scale experiment is to observe the consistency of the residue after a thermolysis operation to determine whether it can be handled after leaving the thermolysis reactor.

Examples 1-4 are performed on biomass containing 60% by weight of P3HP. The α,β-unsaturated carboxylic acid obtained after thermolysis is acrylic acid (AA).

The step of mixing the biomass containing the P3HP with the inhibitor is performed by introducing 2 g of this biomass and 0 or 20 mg of MEHQ into a 50-ml two-neck round-bottom flask equipped with a bar magnet. The medium is stirred using a magnetic stirrer, so as to spread the inhibitor throughout the solid.

At the start of the experiment, the system is placed under the desired pressure, and the round-bottom flask containing the biomass is then placed in a heating system allowing the desired thermolysis temperature to be established (oil bath or electric heating mantle).

As soon as the thermolysis reactor reaches more than 170 °C, AA vapors are formed, condensing predominantly in the side condenser. After 4 h of heating, the formation of AA vapors in the thermolysis reactor tapers off and the experiment is then stopped. The state of fouling and also the consistency of the thermolysis residue are assessed visually at the end of the experiment.

It is found that addition of the inhibitor allows a very hard solid to be transformed into a pasty and tacky solid which can be upgraded in the invention.

The operating conditions for tests 1-4 are shown in table 1.

TABLE 1

| Test | Inhibitor | Operating pressure (kPa) | Thermolysis residue |
|---|---|---|---|
| 1 | MEHQ | 100 | Viscous pasty solid |
| 2 | MEHQ | 55 | Viscous pasty solid |
| 3 | MEHQ | 20 | Viscous pasty solid |
| 4 | NO | 100 | Very hard, tacky solid |

The results in table 1 demonstrate that the physical state of the residue is dependent on the presence of the inhibitor. The addition of inhibitor enables the residue to become pasty and viscous, whereas it was very hard and tacky without the addition of the latter. This change in consistency of the residue allows easier extraction of the latter when it comes to carrying out the thermolysis continuously.

### Thermogravimetric Analysis Test

The purpose of this test is to provide information regarding the operating conditions required to perform the thermolysis of the P3HA contained in the biomass. In order to better still evaluate the degree of thermolysis, this test was performed with P3HP extracted from the biomass.

10 g of pure P3HP were subjected to thermogravimetric analysis. Thermogravimetric analysis is a technique consisting in measuring the variation in mass of a sample as a function of time, for a given temperature. To do this, for each temperature condition, 1 g of P3HP is placed on the apparatus balance while flushing with nitrogen, and the weight loss over time is then recorded. It is found that the mass loss becomes significant and rapid at and above 170 °C.

From a thermolysis point of view, the least severe operating conditions would be: T° of 180 °C for a residence time of 3 h, to obtain a degree of cracking of over 95%. Table 2 indicates the temperature conditions and times required to obtain different degrees of cracking, from 10% to complete cracking.

TABLE 2

| | Time required for: | | | | | |
|---|---|---|---|---|---|---|
| | 10% | | 50% | | Complete | |
| | cracking | | cracking | | cracking (>95%) | |
| 120 °C | >24 | h | >24 | h | >24 | h |
| 130 °C | 14 | h | >24 | h | >24 | h |
| 140 °C | 6 | h | 24 | h | >24 | h |
| 150 °C | 2 | h | 8 | h | 15 | h |
| 160 °C | 1 | h30 | 6 | h | 10 | h |
| 170 °C | 1 | h | 4 | h | 8 | h |
| 180 °C | 45 | min | 2 | h | 3 | h |
| 190 °C | 30 | min | 1 | h15 | 1 | h45 |
| 200 °C | <30 | min | 45 | min | 1 | h |
| 210 °C | <30 | min | 40 | min | 50 | min |
| 220 °C | <30 | min | 35 | min | 40 | min |

The thermolysis reaction temperature of about 180 °C in the process according to the invention is significantly lower than that used in Example 1 of WO 2016/039618. The tests in the following Examples 5-7 are performed in a laboratory set-up.

A two-necked round-bottomed flask equipped with magnetic stirring was used. The side neck of the round-bottom flask is equipped with a thermometer to monitor the reaction temperature. The upper neck of the round-bottom flask is equipped with a separation bridge leading to a water-cooled side condenser, itself leading to a receiver consisting of a second 50 ml round-bottom flask. An additional branch line allows the assembly to be pressurized by means of a diaphragm vacuum pump.

### Example 5 (Comparative): Use of a Biomass Containing 60% P3HP without Addition of Inhibitor and without Catalyst

2.02 g of biomass containing 60% of P3HP are placed in a 25-ml two-neck round-bottom flask equipped with magnetic stirring. The round-bottom flask equipped with a separation bridge is placed at 20 kPa of pressure by means of a diaphragm vacuum pump. The round-bottom flask is heated at 200 °C for 4 h. The vapors generated are condensed using a water-cooled side condenser to obtain 1.06 g of acrylic acid, which corresponds to 87% yield. The solid obtained after cracking remains bonded to the walls of the two-necked round-bottomed flask. It is very difficult to remove from the two-neck round-bottom flask. Solid particles are found in the top of the round-bottom flask and on the side condenser.

### Example 6 (According to the Invention): Use of a Biomass Containing 60% of P3HP by Addition of 1% of 4-Methoxyphenol (MEHQ) without Catalyst

2.11 g of biomass containing 60% of P3HP are placed in a 25-ml two-neck round-bottom flask equipped with magnetic stirring. 0.021 g of MEHQ are added to the round-bottom flask and mixed with the biomass. The round-bottom flask equipped with a separation bridge is placed at 20 kPa of pressure by means of a diaphragm vacuum pump. The round-bottom flask is heated at 200 °C for 4 h. The vapors generated are condensed using a water-cooled side condenser to obtain 1.21 g of acrylic acid, which corresponds to 95% yield. The solid obtained after cracking remains compact and readily detaches from the walls of the two-neck round-bottom flask. This solid breaks up easily with a spatula.

### Example 7 (According to the Invention): Use of a Biomass Containing 60% of P3HP by Addition of 5% of 4-Methoxyphenol (MEHQ)

2.19 g of biomass containing 60% of P3HP are placed in a 25-ml two-neck round-bottom flask equipped with magnetic stirring. 0.117 g of MEHQ are added to the round-bottom flask and mixed with the biomass. The round-bottom flask equipped with a separation bridge is placed at 20 kPa of pressure by means of a diaphragm vacuum pump. The round-bottom flask is heated at 200 °C for 4 h. The vapors generated are condensed using a water-cooled side condenser to obtain 1.27 g of acrylic acid, which corresponds to 96% yield. The solid obtained after cracking remains compact and readily detaches from the walls of the two-neck round-bottom flask and is easily broken up with a spatula.

The results of examples 6 and 7 show that when the process according to the invention is performed (in the presence of inhibitor and without catalyst) the acrylic acid yield reaches 95%, higher than that obtained in comparative example 5. Furthermore, the presence of the inhibitor allows the residue to be readily removed.

1. A process for producing a biobased α,β-unsaturated carboxylic acid from a biomass containing a poly(3-hydroxyalkanoate) (P3HA) and in the absence of catalyst, said process comprising the following steps:
mixing said biomass with at least one polymerization inhibitor, in the solid state to obtain a biomass-inhibitor mixture;
subjecting said biomass-inhibitor mixture to a thermolysis step resulting in a vapor phase comprising α,β-unsaturated carboxylic acid and a solid residue;
separating the vapor phase and the solid phase residue into a gas phase and a solid phase residue;
purifying said gas phase to obtain a purified α,β-unsaturated carboxylic acid; and
treating the solid phase residue.

2. The process as claimed in claim 1, wherein:
mixing the biomass and the at least one polymerization inhibitor in a conveyor mixer comprising several endless screws actuated in a sheath or directly in a thermolysis reactor, wherein the biomass and the at least one polymerization inhibitor is introduced into the conveyor mixer or the thermolysis reactor by means of a pipe or by an endless screw-type conveyor;
thermolysis is carried out at a thermolysis temperature and at a controlled thermolysis pressure in a system suitable for treating the biomass-inhibitor mixture to generate the vapor phase and the solid phase residue;
separating the vapor phase and the solid phase residue is carried out in a gas-liquid separator;
treating the solid phase residue comprises land spreading, combustion or hydrothermal gasification;
the process further comprising:
condensing the gas phase, carried out by successive condensation temperature adjustment by placing one or more condensers in series and separating the resulting gas and liquid phases containing α,β-unsaturated carboxylic acid and contaminants which can be recycled back into the reactor or sent to the purification system;
treating the condensed phase to obtain the α,β-unsaturated carboxylic acid by using one to several distillation columns, firstly, to separate the α,β-unsaturated carboxylic acid from the products heavier than the α,β-unsaturated carboxylic acid and, secondly, to obtain products lighter than the α,β-unsaturated carboxylic acid; and
purifying the resulting α,β-unsaturated carboxylic acid by a liquid/solid separation method or by a gas/liquid separation method.

3. The process as claimed in claim 1, wherein the biomass is pretreated by means of washing, drying or milling operations to yield a biomass containing at least 30% by weight of P3HA.

4. The process as claimed in claim 1, wherein the poly(3-hydroxyalkanoate) contains a 3-hydroxypropionate unit and at least one of the α,β-unsaturated carboxylic acids produced is acrylic acid.

5. The process as claimed in claim 1, wherein the poly(3-hydroxyalkanoate) is poly(3-hydroxypropionate) and the α,β-unsaturated carboxylic acid produced is acrylic acid.

6. The process as claimed in claim 1, wherein the poly(3-hydroxyalkanoate) contains a 3-hydroxybutyrate unit and at least one of the α,β-unsaturated carboxylic acids produced is crotonic acid.

7. The process as claimed in claim 1, wherein the poly(3-hydroxyalkanoate) is poly(3-hydroxybutyrate) and the α,β-unsaturated carboxylic acid produced is crotonic acid.

8. The process as claimed in claim 1, wherein the poly(3-hydroxyalkanoate) contains a 3-hydroxyisobutyrate unit and at least one of the α,β-unsaturated carboxylic acids produced is methacrylic acid.

9. The process as claimed in claim 1, wherein the poly(3-hydroxyalkanoate) is poly(3-hydroxyisobutyrate) and the α,β-unsaturated carboxylic acid produced is methacrylic acid.

10. The process as claimed in claim 1, wherein the at least one polymerization inhibitor is chosen from phenol derivatives, phenothiazine derivatives, nitroxide derivatives and para-phenylenediamine derivatives.

11. The process as claimed in claim 10, wherein the at least one polymerization inhibitor is hydroquinone methyl ether.

12. The process as claimed in claim 2, wherein the thermolysis reactor is chosen from: a conveyor, a mixer-conveyor, a dryer, a rotating drum and a set of heating plates.

13. The process as claimed in claim 2, wherein the thermolysis temperature is between 150 °C and 200 °C.

14. The process as claimed in claim 1, wherein the thermolysis step is performed for two to four hours.

15. The process as claimed in claim 2, wherein the thermolysis pressure is between 15 kPa and 40 kPa.

16. The process as claimed in claim 12, wherein the thermolysis reactor is a device of mixer-conveyor type, for example of paddle dryer type.

17. The process as claimed in claim 12, wherein the thermolysis reactor is a device consisting of hollow plates heated by a heat-transfer fluid circuit.

18. The process as claimed in claim 12, wherein the thermolysis reactor is a screw conveyor.

19. The process as claimed in claim 12, wherein the mixing the biomass and the at least one polymerization inhibitor and the thermolysis step may be performed consecutively or simultaneously.

20. The process as claimed in claim 3, wherein the biomass contains at least 50% by weight of P3HA.
